(19) United States
(12) Patent Application Publication
(10) Pub. No.: US 2009/0093521 A1
(43) Pub. Date: Apr. 9, 2009

(54) 2, 5-Bis-Diamine [1,4] Benzoquinone-Derivatives

(76) Inventors: Maria Laura Bolognesi, Bologna (IT); Rita Banzi, Pieve Di Cento (IT); Anna Minarini, Bologna (IT); Carlo Melchiorre, Bologna (IT)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 11/917,560

(22) PCT Filed: Jun. 13, 2005
(86) PCT No.: PCT/IB06/01561

§ 371 Date: Dec. 16, 2008

(30) Foreign Application Priority Data
Jun. 14, 2005 (IT) BO2005A 000402

(51) Int. Cl. A61K 31/135; C07C 211/33; C07D 409/12; C07D 401/12; A61P 25/28; A61K 31/444; A61K 31/381

(52) U.S. Cl. 514/332; 564/306; 549/59; 546/264; 514/444; 514/655

(57) ABSTRACT

Synthesis of 2,5-bis-diamine-[1,4]benzoquinonic derivatives having the general formula (I), products and intermediates of said synthesis; the synthesis involves the use of p-benzoquinones having the general formula (IX) and diamines having the general formula (XI).

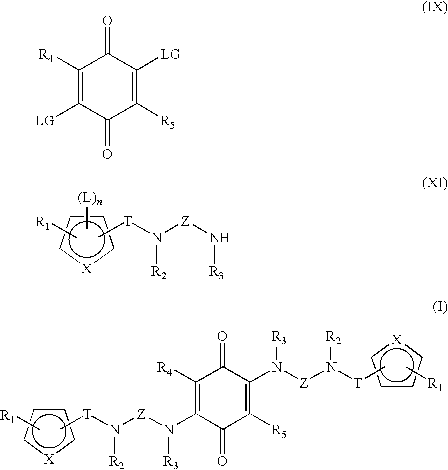

### TECHNICAL BACKGROUND

The present invention relates to a synthesis method for organic compounds, in particular for 2,5-bis-diamine-[1,4]benzoquinone derivatives, intermediates and products of said method. Furthermore, the present invention relates to products for the treatment of Alzheimer's disease and for the production of pharmaceutical preparations for the treatment of Alzheimer's disease.

### PRIOR ART

Alzheimer's disease is a neurodegenerative syndrome generally linked with aging, and which provokes in patients a progressive deterioration in cognitive and behavioural functions. The causes of the great majority of cases of Alzheimer's disease are basically still unknown. It is also for this reason that till today there are still no therapeutic treatments able to arrest the progress of the disease, even though certain pharmaceuticals were recently introduced onto the market, mainly directed at controlling cognitive symptoms. These pharmaceuticals—tacrine (Cognex®), donepezil (Aricept®) rivastigmine (Exelon®) and galantamine (Reminyl®)—have the same mechanism of action in common, consisting in the inhibition of acetylcholine esterase (AChE).

In the field of pharmaceutical products for the treatment of Alzheimer's disease—patent application PCT/IT03/00227 succeeded in identifying a new family of 2,5-bis-diamine-[1,4]benzoquinone derivatives, which, among other properties, showed relatively high activity in treating Alzheimer's disease in mammals.

However, the common synthesis methods for producing 2,5-bis-diamine-[1,4]benzoquinone derivatives have not resulted as being completely satisfactory up till now, as they are not very versatile and do not produce a very high yield. In particular, the well-known reaction of diamine attack on quinone (described in general terms on page 13, lines 2-5 of patent PCT/IT03/00227) has a yield of approximately 30%; the well-known alkylation reaction (described in general terms on page 11, line 18-page 12, line 5 of patent PCT/IT03/00227) reduces its own yield considerably increasing the quantity of starting products and requires chromotographic separation, which is often a disadvantage at industrial level; the intermediates composed of a protected diamine (as described in general terms on page 11, line 4 of patent PCT/IT03/00227) has current commercial costs that are relatively high.

### AIM OF THE INVENTION

The aim of the present invention is to provide synthesis methods for 2,5-bis-diamine-[1,4]benzoquinone which will help overcome the aforementioned problems, at least partially, and which are easy and economical to perform at the same time.

According to the statements above, it has also been detected that as well as the desire to find new synthetic methods to produce recognised derivatives, there is also a large need for making new medications available for the treatment of Alzheimer's disease.

A further aim of the present invention is to produce substances which can be used to advantage in the treatment of Alzheimer's disease.

In accordance with the present invention the followings are provided: synthesis methods for 2,5-bis-diamine-[1,4]benzoquinone derivatives, 2,5-bis-diamine-[1,4]benzoquinone derivatives, 2,5-bis-diamine-[1,4]benzoquinone derivatives for use as medicaments, and uses of said derivatives according to the following appended independent claims, and preferably, in any one of the claims directly or indirectly subordinate to the independent claims.

In the present text the term “alkoxy group Cₓ-C_{y}” refers to an alkyl having from X to Y carbon atoms and linked to the remaining part of the molecule by means of an oxygen atom.

Certain compounds in the present text can present one or more asymmetrical centres; these compounds can therefore be produced as (R)— or (S)— stereoisomers or as a mixture of them. Unless specified otherwise, the compounds identified in the present text are to be understood as comprising both the isomers taken individually, as well as their mixtures, racemic or other types. Methods used to determine the stereochemistry and separation of the stereoisomers are already known in prior art (for example, refer to Chapter 4 of “Advanced Organic Chemistry”, 4^{th} edition L. March, John Wiley and Sons, New York, 1992).

Certain compounds in the present text can present tautomeric and diastereomeric phenomena; unless specified otherwise, these compounds are to be understood as comprising both tautomers and/or diastereomers taken both individually and as their mixture.

A method for the synthesis of a 2,5-bis-diamine-[1,4]benzoquinone derivative is provided according to a first aspect of the present invention, having the following general formula (I):

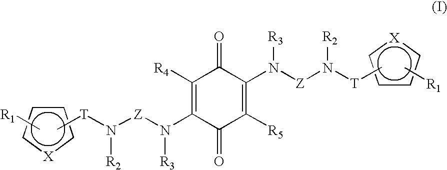

wherein R₁ represents a substituent chosen in the group consisting of:
a hydrogen atom,
a saturated or unsaturated linear or branched Alkyl group with one to five carbon atoms, and
a substituent having an inductive electron withdrawing effect;

R₂ and R₃ represent, each independently from one another, a hydrogen or a saturated or unsaturated linear or branched alkyl group having from one to five carbon atoms; R₄ and R₅ represent, each independently from one another, a substituent chosen in the group consisting of:
hydrogen,
a saturated or unsaturated linear or branched alkyl group with one to five carbon atoms, and
a halogen,
   X represents a radical chosen in a group consisting of:: —HC═CH—, —HC═N—, —S—, —O—, and —NH—; T represent a saturated or unsaturated linear or branched alkyl group with one to four carbon atoms; Z represents a saturated or unsaturated linear or branched alkyl group with two to thirteen carbon atoms. Preferably, X represents the radical —HC═CH—, —HC═N— or —S—.

The method involves a nucleophilic substitution step, wherein, on a p-benzoquinone having a general formula (IX):

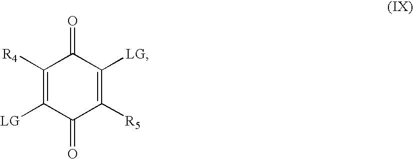

wherein LG represents a leaving group having an inductive electron withdrawing effect, a substitution is carried out with a first compound having a general formula (VIII);

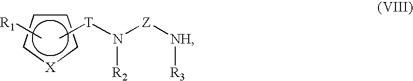

in order to obtain the 2,5-bis-diamine-[1,4]benzoquinone derivative having a general formula (I).

Preferably, R₁ represents a substituent chosen in the group consisting of:
a hydrogen,
a substituent having an inductive electron withdrawing effect.

According to preferred embodiments, R₁ represents a substituent chosen in the group consisting of:
a hydrogen,
a halogen,
NO₂, and
an alkoxy C₁-C₃;

R₂ and R₃ represent each independently from one another, a hydrogen or a saturated or unsaturated linear alkyl group having from one to four carbon atoms; R₄ and R₅ represent, each independently from one another, a substituent chosen in the group consisting of:
hydrogen,
a saturated or unsaturated linear or branched alkyl group having from one to five carbon atoms,
a halogen,
   T represents a saturated or unsaturated linear alkyl having from one to three carbon atoms;
   Z represents a saturated or unsaturated linear alkyl having from two to twelve carbon atoms.

According to even further preferred embodiments, R₁ represents a substituent chosen in the group consisting of:
a hydrogen,
a halogen,
a saturated or unsaturated linear or branched alkyl group from one to four carbon atoms, and
an alkoxy C₁-C₃;
   R₂ and R₃ represent, each independently from one another, a hydrogen or a saturated linear alkyl group having from one to two carbon atoms; R₄ and R₅ represent, each independently from one another, a substituent chosen in the group consisting of:
a hydrogen,
a saturated linear or branched alkyl group having from one to four carbon atoms,
a halogen,
   X represents the radical —HC═CH— or the radical —O—;
   T represents the radical —CH₂—; and
   Z represents a saturated linear alkyl having from two to seven carbon atoms.

According to yet even further preferred embodiments, R₃ represents a hydrogen; R₄ and R₅ represent, each independently from one another, a substituent chosen in the group consisting of:
a hydrogen,
a saturated linear alkyl group having from one to two carbon atoms,
a branched alkyl group having from three to four carbon atoms,
a halogen,
   Z represents a saturated linear alkyl having from two to seven carbon atoms.

Preferably, R₁ is in position 2 in relation to T; X represents the radical —HC═CH—; R₄ and R₅ each represent, a respective hydrogen.

More preferably, R₄ and R₅ each represent a respective halogen, preferably a fluorine or a bromine; it can be seen that synthesis of compounds wherein R₄ and R₅ are equal with each other, is simpler.

Preferably, R₂ represents a saturated linear alkyl having from one to two carbon atoms; R₃ represents hydrogen; R₁ represents an alkoxy group C₁-C₂

Furthermore, the following are the preferred embodiments: R₁ represents a methoxy group, R₂ represents an ethyl, R₃, R₄ and R₅ each represent a relative hydrogen, Z represents a propyl, T represents a methyl, X represents the radical —HC═CH—; R₁ represents a methoxy group, R₂ represents an ethyl, R₃, R₄ and R₅ each represent a relative hydrogen, Z represents a butyl, T represents a methyl, X represents the radical —HC═CH—; R₁ represents a methoxy group, R₂ represents an ethyl, R₃, R₄ and R₅ each represent a relative hydrogen, Z represents a butyl, T represents a methyl, X represents the radical —HC═CH—; R₁ represents a methoxy group, R₂ represents an ethyl, R₃, R₄ and R₅ each represent a relative hydrogen, Z represents a pentyl, T represents a methyl, X represents the radical —HC═CH—; R₁ represents a methyl, R₂ represents an ethyl, R₃, R₄ and R₅ each represent a relative hydrogen, Z represents a hexyl, T represents a methyl, X represents the radical —HC═CH—; R₁ represents a methoxy group, R₂ represents an ethyl, R₃, R₄ and R₅ each represents a relative hydrogen, Z represents a hexyl, T represents a methyl, X represents the radical —S—; R₁ represents a methoxy group, R₂ represents an ethyl, R₃, R₄ and R₅ each represent, a relative hydrogen, Z represents a hexyl, T represents a methyl, X represents the radical —HC═N—; R₁ represents a methoxy group, R₂ represents an ethyl, R₃ represents a hydrogen, R₄ and R₅ each represent, a relative fluorine, Z represents a hexyl, T represents a methyl, X represents the radical —HC═CH—; R₁ represents a methoxy group, R₂ represents an ethyl, R₃ represents a hydrogen, R₄ and R₅ each represent a relative bromine, Z represents a hexyl, T represents a methyl, X represents the radical —HC═CH—.

According to preferred embodiments, LG represents an alkoxy group; preferably, LG represents an alkoxy group C₁-C₅, more preferably, LG represents an alkoxy group C₁-C₃; even more preferably LG represents a methoxy group.

According to further preferred embodiments, LG represents a halogen; preferably LG represents a halogen chosen in the group consisting of: Fluorine, Bromine.

According to preferred embodiments, the nucleophilic substitution step occurs in the presence of an alcoholic solvent; preferably, the alcoholic solvent is ethanol. To activate the nucleophilic substitution in the most correct manner it is preferable to work at a temperature between 50° C. and 65° C., in other words under reflux ethanol conditions.

Preferably, the intermediate compound (VIII) as defined previously, is obtained through the hydrolysis of an intermediate compound having a general formula (VII):

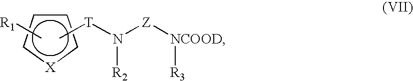

wherein D represents a benzyl (En) or another protective group that is substantially stable in a base environment; hydrolysis occurs in an acid environment.

According to certain embodiments, the intermediate compound having the general formula (VII) can be in turn obtained by using well-known methods such as that described in patent application PCT/IT03/00227, the contents of which are incorporated in this text for reference.

According to alternative and preferred embodiments, an intermediate compound having a general formula (V):

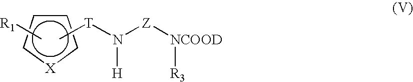

is made to react in the presence of a reducer, with a compound having a general formula (VI):

R₂=O (VI),

in order to obtain the intermediate compound having a general formula (VII) during the addition step.

Preferably, the reducer is NaBH₃CN.

The addition step occurs in a particularly clean manner if performed in an alcoholic solvent.

It is possible to purify the intermediate compound having the general formula (VII) at the end of the addition step through extraction using a substantially apolar solvent, preferably n-hexane or petroleum ether. It can be seen that when applied industrially, purification through extraction presents considerable advantages compared to chromatographic purification.

Alternatively, the intermediate compound having a general formula (V) can be made to react in the presence of a reducer, with a compound having a general formula (XIII):

R₂COOH (XIII),

in order to obtain the said second intermediate compound having a general formula (VII). In this case, preferably, the reducer is NaBH₄ and the addition step occurs in tetrahydrofuran (THF) as the solvent.

According to preferred embodiments, the intermediate compound having a general formula (V) is obtained as a result of a further addition step, during which a protected carbamic (amine-alkyl) acid having a general formula (III):

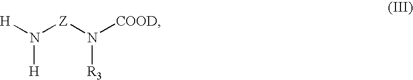

is added to a compound having a general formula (IV):

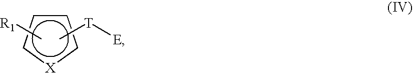

wherein E represents a residue chosen in the group consisting of: ═O, —Cl, —Br, and —I; with the proviso that where E represents ═O the method comprises a reduction step to obtain said third intermediate compound having a general formula (V). Preferably, E represents ═O.

Although the protected (amine-alkyl)-carbamic acid having a general formula (III) is normally available on the market, it is preferable to obtain it by using a protection step, which involves making a diamine having a general formula (II):

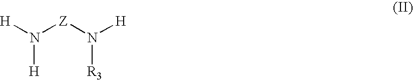

react with benzylchloroformiate in order to obtain the protected (amine-alkyl)-carbamic acid having a general formula (III) wherein D represents a benzyl (Bn).

In relation to this aspect it is important to emphasise that the protected (amine-alkyl)-carbamic acid having a general formula (III), wherein D represents a benzyl (Bn), currently has a cost of approximately 52 euro per gram; on the other hand, based on the current price of starting materials for the protection reaction, it has been calculated that it will cost approximately 100 euros for 100 grams of the protected (amine-alkyl)-carbamic acid having a general formula (III), wherein D represents a benzyl (Bn), obtained using the protection step described above.

Preferably, during the protection step, the molar ratio between the diamine having a general formula (II) and the benzylchlorofomiate is approximately three to one.

According to a further embodiment of the present invention, a method is provided for the synthesis of an intermediate compound having a general formula (VII) as defined previously, comprising an addition step as defined above, among the compounds having a general formula (V) and respectively (VI).

According to further aspects of the present invention, a 2,5-bis-diamine-[1,4]benzoquinone derivative is provided having a general formula (X):

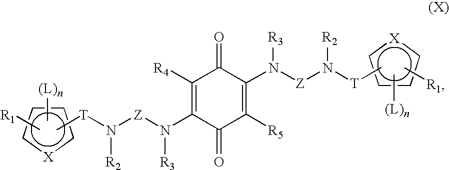

wherein R₁, R₂, R₃, R₄, R₅, X, T and Z are defined as above, L represents a halogen and n is an integer greater than or equal to 1, and less than or equal to 3; and a method for the synthesis of a 2,5-bis-diamine-[1,4]benzoquinone derivative having a general formula (I) as defined above, using the 2,5-bis-diamine-[1,4]benzoquinone derivative having a general formula (X). In particular the method comprises a reduction step of the 2,5-bis-diamine-[1,4]benzoquinone derivative having a general formula (X); preferably, the reduction is obtained through catalytic hydrogenation. According to preferred embodiments, L represents a bromine and n is equal to 2.

According to a further aspect of the present invention, the 2,5-bis-diamine-[1,4]benzoquinone derivative having a general formula (X) is obtained using a synthesis method comprising a nucleophilic substitution step, during which, on a p-benzoquinone having a general formula (IX):

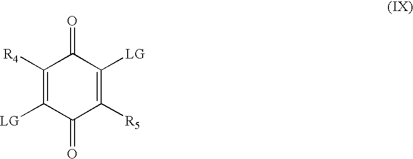

wherein LG represents a leaving group having an inductive electron withdrawing effect, a substitution is performed with a first intermediate compound having a general formula (XI):

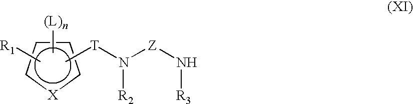

Preferably, LG is defined as above and the nucleophilic substitution step is performed in a manner similar to that described for the synthesis of the 2,5-bis-diamine-[1, 4]benzoquinone derivative having a general formula (I).

It is possible to obtain the intermediate compound having a general formula (XI) using a synthesis method similar to that used to produce the intermediate compound (VIII).

In agreement with a further aspect of the present invention, a 2,5-bis-diamine-[1,4]benzoquinone derivative is produced, having a general formula (XII):

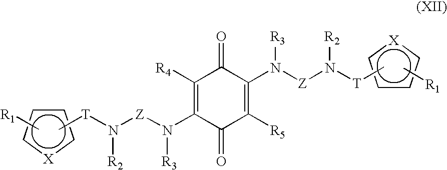

wherein R₄ and R₅ each represent a respective halogen; R₁, R₂, R₃, X, T and Z being defined as above.

According to preferred embodiments R₄ and R₅ each represent a respective halogen chosen in the group consisting of: fluorine, chlorine and bromine; preferably, fluorine and bromine.

According to a further aspect of the present invention, a 2,5-bis-diamine-[1,4]benzoquinone derivative is provided, having a general formula (XII) for use as a medication.

According to a further aspect of the present invention, a 2,5-bis-diamine-[1,4]benzoquinone derivative is provided having a general formula (XII) for the treatment of Alzheimer's disease.

Further characteristics of the present invention will be made clear from the following description of several examples provided in a non limiting manner purely to illustrate the principle.

In particular, it can be seen that certain compounds can be synthesised using methods described in the examples provided in the patent application, PCT/IT03/00227, whose contents have been incorporated herein for reference.

### EXAMPLES 1-9

The Examples from 1 to 9 follow the synthetic layout shown below.

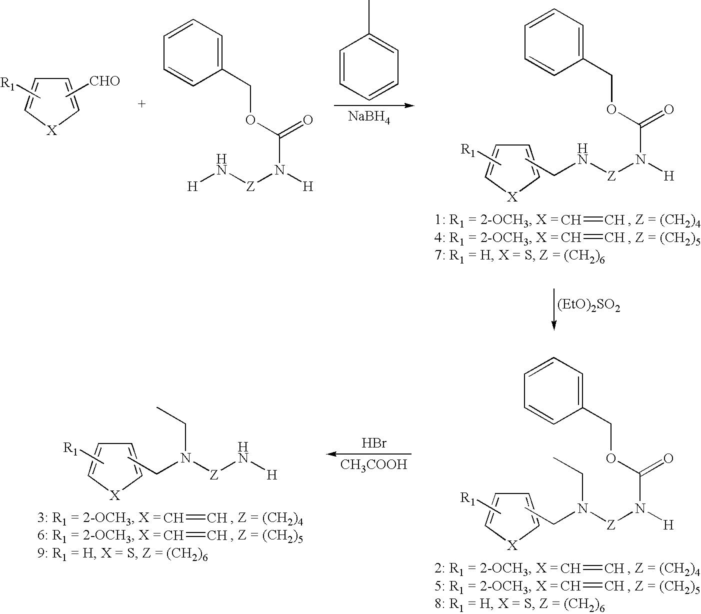

### EXAMPLE 1

### Synthesis of [4-(2-methoxy-benzylamine)-butyl]-carbamic acid benzyl ester (1)

The (4-amine-butyl)-carbamic acid benzyl ester (commercial type) (1.20 g; 5.40 mmol) solubilized in toluene (30 ml) to which is added 2-methoxybenzaldehyde (0.76 g; 5.9 mmol). It is heated at reflux with a Dean-Stark apparatus for 6 hours and then left to cool, the toluene is evaporated under vacuum, and the residue is collected using EtOH (30 ml). NaBH₄ (0.20 g; 5.4 mmol) is added, cooling with H₂O and ice and left under agitation at room temperature overnight. The solution is acidified up to pH=2 using HCl 3N, the solvent is evaporated and the residue is collected using H₂O. The aqueous phase is washed several times with ethyl ether basified with NaOH 40% and finally extracted with CHCl₃ (3×30 ml). The combined and anhydrified organic extracts are evaporated under vacuum to obtain the compound 1 in the form of transparent oil. Yield 97%.

¹H NMR (free base, CDCl₃) δ: 1.51-1.63 (m, 4H+1H exchangeable with D₂O); 2.62 (t, 2H); 3.18-3.29 (m, 2H); 3.79 (s, 2H); 3.83 (s, 3H); 5.12 (s, 2H); 5.33 (broad s, 1H exchangeable with D₂O); 6.83-6.79 (m, 2H); 7.20-7.31 (m, 2H); 7.35-7.42 (m, 5H).

### EXAMPLE 2

### Synthesis of {4-[ethyl-(2-methoxy-benzyl)-amine]-butyl}-carbamic acid benzyl ester (2)

A solution of diethylsulfate (1.2 ml; 9.3 mmol) in 30 ml of toluene is added dropwise to a solution of amine 1(1.60 g; 4.68 mmol) in toluene (80 ml). It is left under agitation at reflux for 6 hours at room temperature overnight, then the toluene is decanted and the residue is washed repeatedly with petroleum ether. The oil thus obtained is collected using H₂O, basified with NaOH 2N and extracted with CHCl₃ (3×30 ml). The combined and anhydrified organic extracts, are evaporated under vacuum to obtain a residue purified through flash chromatography with mobile phase with catalytic CH₂Cl₂/petroleum ether/EtOH/NH₃ aqueous 28% (7:2.5:0.5:0.04). Yield 51%; yellow oil.

¹H NMR (free base, CDCl₃) δ: 1.18 (t, 3H); 1.53-1.61 (m, 4H); 2.45-2.62 (m, 4H); 3.17-3.28 (m, 2H); 3.62 (s, 2H); 3.83 (s, 5H); 5.18 (s, 2H); 5.38 (broad s, 1H exchangeable with D₂O); 6.84-7.01 (m, 2H); 7.20-7.25 (m, 1H); 7.33-7.45 (m, 5H).

### EXAMPLE 3

### Synthesis of N¹-ethyl-N¹-(2-methoxy-benzyl)-1,4 butandiamine (3)

The compound 2 (0.88 g; 2.37 mmol) is treated in CH₃COOH (25 ml) with HBr 30% in CH₃COOH (5 ml). It is left under agitation at room temperature overnight and then, cooling it with H₂O and ice, ethyl ether is added until the precipitation is complete. The precipitate formed in this manner is then washed with ethyl ether (3×) and collected using H₂O. The aqueous solution is basified with NaOH tablets and the product is extracted with CHCl₃ (3×50 ml). The combined and anhydrified organic extracts are evaporated under vacuum to provide yellow oil. Quantitative yield.

¹H NMR (free base, CDCl₃) δ: 1.15 (t, 3H); 1.27 (broad s, 2H exchangeable with D₂O); 1.39-1.62 (m, 4H); 2.42-2.60 (m, 4H); 2.68 (t, 2H); 3.60 (s, 2H); 3.82 (s, 3H); 6.82-6.98 (m, 2H); 7.19-7.28 (m, 1H); 7.41-7.45 (m, 1H).

### EXAMPLE 4

### Synthesis of [5-(2-methoxy-benzylamine)-pentyl]-carbamic acid benzyl ester (4)

This is synthesised from (5-amine-pentyl)-carbamic benzyl acid ester (commercial type) (0.50 g; 2.12 mmol) following the procedure described in Example 1. Yield 92%; transparent oil.

¹H NMR (free base, CDCl₃) δ: 1.23-1.75 (m, 6H+1H exchangeable with D₂O); 2.60 (t, 2H); 3.20 (q, 2H); 3.79 (s, 2H); 3.83 (s, 3H); 4.83 (broad s, 1H exchangeable with D₂O); 5.7 (s, 2H); 6.82-6.98 (m, 2H); 7.20-7.42 (m, 7H).

### EXAMPLE 5

### Synthesis of {5-[ethyl-(2-methoxy-benzyl)-amine]-pentyl}-carbamic acid benzyl ester (5)

This is obtained by treating the amine 4 (0.70 g; 1.96 mmol) with diethylsulfate as described in Example 2. The raw substance obtained is purified through flash chromatography with mobile phase using CH₂Cl₂/MeOH (9.25:0.75). Yield 60%; yellow oil.

¹H NMR (free base, CDCl₃) δ: 1.21-1.82 (m, 9H); 2.80-3.07 (m, 4H); 3.18 (q, 2H); 3.81 (s, 3H); 4.08 (s, 2H); 5.08 (s, 2H); 5.42 (broad s, 1H exchangeable with D₂O); 6.83-7.00 (m, 2H); 7.23-7.38 (m, 5H); 7.41-7.50 (m, 2H).

### EXAMPLE 6

### Synthesis of N¹-ethyl-N¹-(2-methoxy-benzyl)-1,5 pentandiamine (6)

This was obtained by treating the compound 5 (0.45 g; 1.17 mmol) with HBr 30% in CH₃COOH as described in Example 3. Yield 70%; yellow oil.

¹H NMR (free base, CDCl₃) δ: 1.08 (t, 3H); 1.23-1.62 (m, 6H+2H exchangeable with D₂O); 2.40-2.75 (m, 6H); 3.60 (s, 2H); 3.82 (s, 3H); 6.82-6.98 (m, 2H); 7.18-7.27 (m, 1H); 7.39-7.46 (m, 1H).

### EXAMPLE 7

### Synthesis of {6-[(thiophen-2-ylmethyl)-amine]-hexyl}-carbamic acid benzyl ester (7)

This is synthesised from (6-amine-hexyl)-carbamic acid benzyl ester (1.00 g; 4 mmol) and 2-thiophencarboxaldehyde as described in Example 1. Yield 90%; yellow oil.

¹H NMR (free base; CDCl₃) δ: 1.32-1.41 (m, 4H); 1.43-1.58 (m, 4H); 2.66 (t, 2H); 3.22 (q, 2H); 4.01 (s, 2H); 4.83 (broad s, 1H exchangeable with D₂O), 5.16 (s, 2H); 6.95-7.02 (m, 2H); 7.25-7.30 (m, 1H); 7.32-7.41 (m, 5H).

### EXAMPLE 8

### Synthesis of [6-(ethyl-thiophen-2-ylmethyl-amine)-hexyl]-carbamic acid benzyl ester (8)

This was obtained from the compound 7 (1.2 g; 3.45 mmol) as described in Example 2. Yield 70%; yellow oil.

¹H NMR (free base; CDCl₃) δ: 1.10 (t, 3H); 1.28-1.60 (m, 8H); 2.41-2.62 (m, 4H); 3.22 (q, 2H); 3.83 (s, 2H); 4.78 (broad s, 1H exchangeable with D₂O); 5.18 (s, 2H); 6.88-7.01 (m, 2H); 7.22-7.28 (m, 1H); 7.35-7.43 (m, 5H).

### EXAMPLE 9

### Synthesis of N¹-ethyl-N¹-thiophen-2-ylmethyl-hexane-1,6-diamine (9)

This is obtained by treating the compound 8 (0.85 g; 2.27 mmol) with HBr as described in Example 3. Yield 80%; yellow oil.

¹H NMR (free base; CDCl₃) δ: 1.12 (t, 3H); 1.32-1.58 (m, 8H+2H exchangeable with D₂O); 2.48 (t, 2H); 2.58 (q, 2H); 2.75 (t, 2H); 3.83 (s, 2H); 6.92-7.02 (m 2H); 7.25-7.32 (m, 1H).

### EXAMPLES 10-18

The Examples from 10 to 18 follow the synthetic layout shown below.

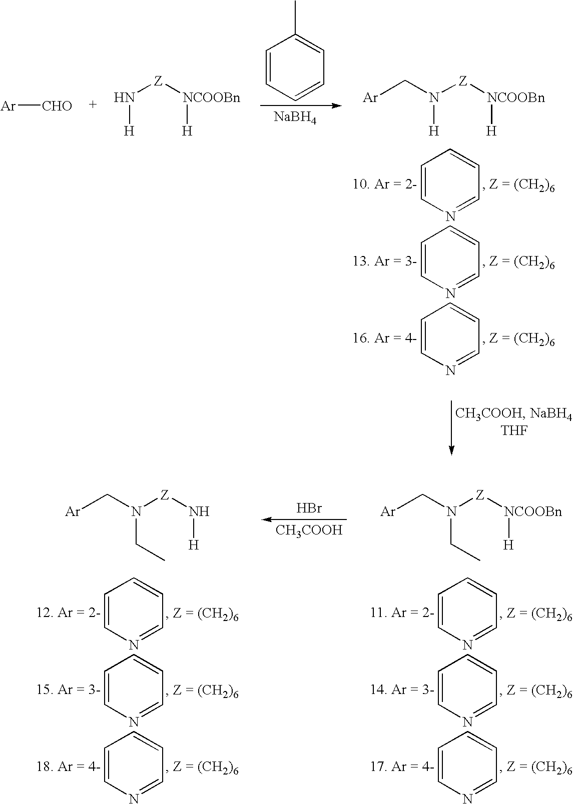

### EXAMPLE 10

### Synthesis of {6[(pyridin-2-ylmethyl)-amine]-hexyl}-carbamic acid benzyl ester (10)

This is obtained from the (6-amine-hexyl)-carbamic acid benzyl ester (4.00 g, 16 mmol) following the same procedure described in Example 1. Yield 55%; yellow oil.

¹H NMR (free base; CDCl₃) δ: 1.28-1.60 (m, 8H); 1.78 (broad s, 1H exchangeable with D₂O); 2.64 (t, 2H); 3.18 (g, 2H); 3.92 (s, 2H); 4.82 (broad s, 1H exchangeable with D₂O); 5.15 (s, 2H); 7.14-7.22 (m, 1H); 7.28-7.40 (m, 6H); 7.61-7.72 (m, 1H); 8.58-8.61 (m, 1H).

### EXAMPLE 11

### Synthesis of [6-(ethyl-pyridin-2-ylmethyl-amine)-hexyl]-carbamic acid benzyl ester (11)

A suspension of the compound 10 (2.00 g; 5.86 mmol) in THF (20 ml) is treated with icy CH₃COOH (10.1 ml). While cooling this with H₂O and ice, small portions of NaBH₄ (0.80 g; 21 mmol) are added. It is left under agitation at 60° C. and under a flow of N₂ for 5 hours. The residue thus obtained is collected using H₂O, acidified up to a pH=2 and washing is performed using ether (2×30 ml). Lastly, the aqueous solution is basified with NaOH tablets, extracted with CH₂Cl₂ (3×50 ml). The anhydrified and evaporated extracts provide a raw substance that is purified through flash chromatography with mobile phase using EtOAc/toluene/EtOH/NH₃ aqueous 28% (7:3:0.5:0.05). Yield 40%; yellow oil.

¹H NMR (free base; CDCl₃) δ: 1.02 (t, 3H); 1.19-1.56 (m, 8H); 2.38-2.58 (m, 4H); 3.15 (q, 2H); 3.63 (s, 2H); 5.05 (s, 2H); 5.09 (broad s, 1H exchangeable with D₂O); 7.02-7.12 (m, 1H); 7.21-7.38 (m, 5H); 7.40-7.48 (m, 1H); 7.52-7.64 (m, 1H); 8.42-8.46 (m, 1H).

### EXAMPLE 12

### Synthesis of N¹-ethyl-N¹-pyridin-2-ylmethyl-hexane-1,6-diamine (12)

This is obtained from the compound 11 (0.70 g; 2.02 mmol) as described in Example 3. Yield 75%; transparent oil.

¹H NMR (free base; CDCl₃) δ: 0.97 (t, 3H); 1.17-1.45 (m, 8H+2H exchangeable with D₂O); 2.35-2.62 (m, 6H); 3.61 (s, 2H); 7.00-7.18 (m, 1H); 7.38-7.43 (m, 1H); 7.52-7.63 (m, 1H); 8.41-8.44 (m, 1H).

### EXAMPLE 13

### Synthesis of {6-[(pyridin-3-ylmethyl)-amine]-hexyl}-carbamic acid benzyl ester (13)

This is obtained from the (6-amine-hexyl)-carbamic acid benzyl ester (4.00 g, 16 mmol) following the same procedure described in Example 1. Yield 50%; yellow oil.

¹H NMR (free base; CDCl₃) δ: 1.25-1.41 (m, 4H+ 1H exchangeable with D₂O); 1.43-1.58 (m, 2H); 1.61-1.78 (m, 2H); 2.73 (t, 2H); 3.18 (q, 2H); 3.98 (s, 2H); 5.02 (broad s, 1H exchangeable with D₂O); 5.12 (s, 2H); 7.28-7.40 (m, 5H); 7.75-7.78 (m, 1H); 7.92-7.99 (m, 1H); 8.55-8.62 (m, 2H).

### EXAMPLE 14

### Synthesis of [6-(ethyl-pyridin-3-ylmethyl-amine)-hexyl]-carbamic acid benzyl ester (14)

The compound is obtained from compound 13 (2.00 g; 5.86 mmol) following the procedure described in Example 11. Yield 40%; yellow oil.

¹H NMR (free base; CDCl₃) δ: 1.02 (t, 3H); 1.19-1.57 (m, 5H); 2.32-2.58 (m, 4H); 3.18 (q, 2H); 3.56 (s, 2H); 5.02-5.10 (m, 2H+1H exchangeable with D₂O); 7.21-7.24 (m, 1H); 7.28-7.38 (m, 5H); 7.61-7.68 (m, 1H); 8.42-8.58 (m, 1H).

### EXAMPLE 15

### Synthesis of N¹-ethyl-N¹-pyridin-3-ylmethyl-hexane-1,6-diamine (15)

This is obtained from the compound 14 (0.75 g; 2.17 mmol) as described in Example 3. Yield 75%; transparent oil

¹H NMR (free base, CDCl₃) δ: 0.95 (t, 3H); 1.12-1.42 (m, 8H+2H exchangeable with D₂O); 2.23-2.45 (m, 4H); 2.58 (t, 2H); 3.42 (s, 2H); 7.11-7.20 (m, 1H); 7.52-7.61 (m, 1H); 8.38-8.46 (m, 1H).

### EXAMPLE 16

### Synthesis {6-[(pyridin-4-ylmethyl)-amine]-hexyl}-carbamic acid benzyl ester (16)

This is obtained from the (6-amine-hexyl)-carbamic acid benzyl ester (4.00 g, 16 mmol) following the same procedure described in Example 1. Yield 60%; yellow oil.

¹H NMR (free base; CDCl₃) δ: 1.21-1.60 (m, 8H+1H exchangeable with D₂O); 2.58 (t, 2H); 3.12-3.23 (m, 2H); 3.78 (s, 2H); 5.02-5.15 (m, 2H+1H exchangeable with D₂O); 7.18-7.40 (m, 7H); 8.42-8.58 (m, 2H).

### EXAMPLE 17

### Synthesis of [6-(ethyl-pyridin-4-ylmethyl-amine)-hexyl]-carbamic acid benzyl ester (17)

The compound is obtained from the compound 16 (2.00 g; 5.86 mmol) following the procedure described in Example 11. Yield 40%; yellow oil.

¹H NMR (free base; CDCl₃) δ: 1.05 (t, 3H); 1.22-1.58 (m, 8H); 2.39-2.60 (m, 4H); 3.18 (q, 2H); 3.58 (s, 2H); 4.84 (broad s, 1H exchangeable with D₂O); 5.15 (s, 2H); 7.23-7.41 (m, 7H); 8.48-8.60 (m, 2H).

### EXAMPLE 18

### Synthesis of N¹-ethyl-N¹-pyridin-4-ylmethyl-hexane-1,6-diamine (18)

The compound is obtained from the compound 17 (0.85 g; 2.30 mmol) as described in Example 3. Yield 75%; transparent oil.

¹H NMR (free base; CDCl₃) δ: 0.92-0.98 (m, 3H); 1.10-1.25 (m, 8H+2H exchangeable with D₂O); 2.21-2.42 (m, 4H); 2.45-2.60 (m, 2H); 3.38 (s, 2H); 7.11-7.20 (m, 2H); 8.37-8.41 (m, 1H).

### EXAMPLE 19

### Synthesis of {6-[ethyl-(2-methoxy-benzyl)-amine]-hexyl}-carbamic acid benzyl ester (19)

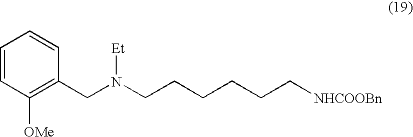

4.2 g of KOH (0.07 mol) are added to a solution of [6-(2-methoxy-benzylamine)-hexyl]-carbamic acid hydrochloric benzyl ester (104 g, 0.26 mol) in methanol (1.2 L), stirred in a mechanical stirrer, and after the base is completely solubilized, acetaldehyde (29 mL, 0.51 mol) is added. The resulting suspension is agitated at room temperature for 15 minutes and then a solution of NaBH₃CN (6.3 g, 0.1 mol) in 50 mL of methanol is added dropwise. When addition is completed, the resulting mixture is agitated overnight. Then KOH (15 g) is added and the suspension obtained is suction filtered on Celite® and concentrated under vacuum at a temperature lower than 45° C. A solid precipitate is obtained, which is then divided between water and CH₂Cl₂ (2×750 mL). The combined organic extracts are anhydrified and concentrated to give the raw compound 19 (106 g) which is purified through flash chromatography. Gradient elution of CHCl₃-CHCl₃ 9.3/EtOH 0.7 provides the pure compound 19 with a yield of 48%

Alternatively the raw product can be purified through extraction with hot petroleum ether. The combined and anhydrified extracts provide the compound 19, which is sufficiently pure for most uses, with a yield of 53%.

¹H NMR (free base; CDCl₃) δ: 1.05 (t, 3H), 1.21-1.32 (m, 4H), 1.40-1.57 (m. 4H), 12.41-2.59 (m, 4H), 3.16 (q, 2H), 3.60 (s, 2H), 3.80 (s, 3H), 4.73 (broad s, 1H exchangeable with D₂O), 5.08 (s, 2H), 6.82-6.94 (m, 2H), 7.18-7.42 (m, 7H).

### EXAMPLES 20-22

The Examples from 20 to 22 follow the synthetic layout shown below.

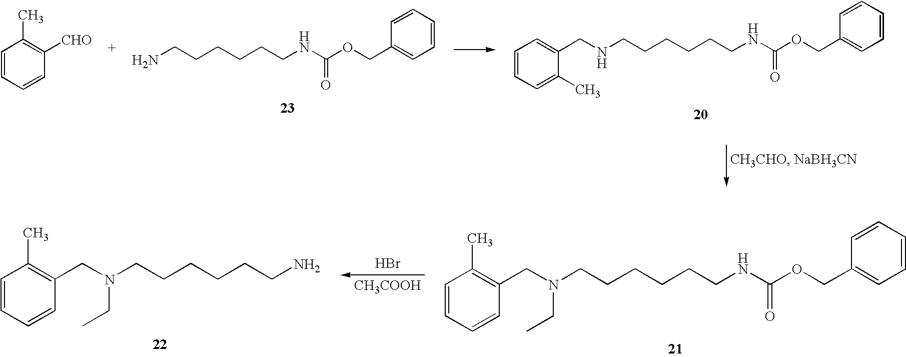

### EXAMPLE 20

### Synthesis of [6-(2-methyl-benzylamine)-hexyl]-carbamic acid benzyl ester (20)

This was obtained from (6-amine-hexyl)-carbamic acid benzyl ester (23) (5.00 g; 20 mmol) following the procedure described in Example 1. Yield 55%; yellow oil.

¹H NMR (free base, CDCl₃) δ: 1.28-1.62 (m, 8H); 1.88 (broad s, 1H exchangeable with D₂O); 2.38 (s, 3H); 2.67 (t, 2H); 3.21 (q, 2H), 3.78 (s, 2H); 5.83 (broad s, 1H exchangeable with D₂O); 5.16 (s, 2H); 7.17-7.21 (m, 3H); 7.27-7.41 (m, 6H).

### EXAMPLE 21

### Synthesis of {6-[ethyl-(2-methyl-benzyl)-amine]-hexyl}-carbamic acid benzyl ester (21)

This compound was prepared following the procedure in Example 19 of [6-(2-methyl-benzylamine)-hexyl]-carbamic acid benzyl ester (20) (3.00 g; 8.47 mmol) and acetaldehyde (0.96 ml; 16.9 mmol). The raw product obtained is purified through flash chromatography with mobile phase using CH₂Cl₂/EtOH (9.7:0.3). Yield 50%; yellow oil. ¹H NMR (free base, CDCl₃) δ: 1.05 (t, 3H); 1.18-1.36 (m, 4H); 1.39-1.55 (m, 4H); 2.35-2.58 (m, 7H); 3.18 (q, 2H); 3.52 (s, 2H); 4; 82 (broad t, 1H exchangeable with D₂O); 5.16 (s, 2H); 7.15-7.21 (m, 3H); 7.23-7.41 (m, 6H).

### EXAMPLE 22

### Synthesis of N¹-ethyl-N¹-(2-methyl-benzyl)-1,6-hexanediamine (22)

This was obtained by treating the compound 21 (1.00 g; 2.67 mmol) with HBr as described in Example 3. Yield 90%; yellow oil.

¹H NMR (free baser CDCl₃) δ: 1.05 (t, 3H); 1.18-1.57 (m, 8H+2H exchangeable with D₂O); 2.28-2.56 (m, 7H); 2.66 (t, 2H); 3.52 (s, 2H); 7.08-7.21 (m, 3H); 7.25-7.38 (m, 1H).

### EXAMPLE 23

### Synthesis of the benzylic ester of (6-amine hexyl)-carbamic acid (23)

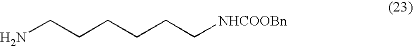

In a three-necked flask equipped with a mechanical stirrer, 1,6-hexandiamine (195 g, 1.68 mol) is solubilized in 300 ml of water containing bromo cresol green as an indicator. At 5° C. methanesulphonic acid is added dropwise, (218 mL, 3.36 mol) until the indicator turns to yellow, and then the resulting solution is diluted with ethanol (500 mL). T is brought to 30° C. and at the same time a solution of benzylchloroformiate (75 mL, 0.52 mol) in 75 mL of dimethoxyethane and a solution of 50% potassium acetate is added dropwise, in order to maintain the pH equal to 4. The additions are completed within 4.5 hours, and then the product is stirred at 30° C. for another hour, followed by further agitation at room temperature overnight. The volatile solvents are evaporated under vacuum and the aqueous solution obtained is filtered to remove the non-substituted derivative that has been thus formed. The filtrate is washed with toluene (5×400 mL), basified with KOH (pH=12) and extracted with toluene (2×350 mL). The organic extracts are washed with water (300 mL), anhydrified and evaporated under vacuum to give 59 g of compound 23 in the form of a waxy solid. Yield 45%.

### EXAMPLE 24

This example describes the synthesis of 2,5-bis-{6-[ethyl-(2-methoxy-benzyl)-amine]-hexyl amine}-[1,4]benzoquinone (24).

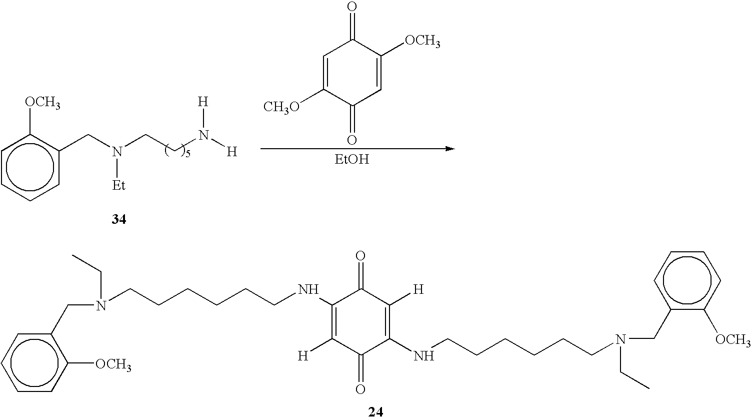

The compound 24 can be prepared from N—ethyl-N¹-(2-methoxy-benzyl)-hexane-1,6-diamine and 2,5-dimethoxyquinone.

N¹-ethyl-N¹-(2-methoxy-benzyl)-hexane-1,6-diamine 34 (12.2 g; 46 mmol) in 150 ml of EtOH is added dropwise to a suspension of 2,5-dimethoxyquinone (3.85 g; 23 mmol) in boiling EtOH (450 ml). The mixture of the reaction becomes progressively clear and red. It is heated to 60° C. for 3 hours and after cooling, it is filtered through a folded filter. The filtrate is concentrated under vacuum to give the compound 24 in the form of a red solid. M.p. 45° C. Quantitative yield.

It was noted that the purity of the compound 24 depends on the purity of the original diamine. If the raw product obtained through the reaction is not sufficiently pure, it can be purified through precipitation of the compound 24 of the ethanol reaction mixture, appropriately filtered and concentrated, with the addition of H₂O. Yield 85%.

Alternatively, a drop chromatography method can be used: elution with CH₂Cl₂ 9.25/MeOH 0.75/NH₄OH 0.075, provides compound 24.

¹H NMR (free base; CDCl₃) δ: 1.04 (t, 6H), 1.14-1.38 (m, 8H), 1.43-1.53 (m, 4H), 1.57-1.65 (m, 4H), 2.41-2.56 (m, 8H), 3.10 (q, 4H), 3.57 (s, 4H), 3.81 (s, 6H), 5.28 (s, 2H), 6.59 (broad t, 21 exchangeable with D₂O), 6.83-6.95 (m, 4H), 7.21 (t, 2H), 7.39 (d, 2H). EI-MS: m/z=632 (M⁺).

### EXAMPLE 25

### Synthesis of 2,5-bis-{2-[ethyl-(2-methoxy-benzyl)-amine]-butylamine}-[1,4]-benzoquinone (25)

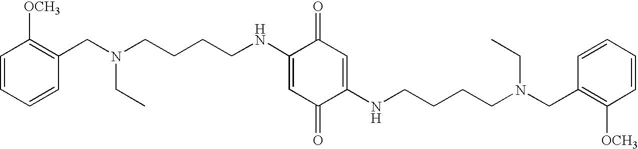

A solution of N1-Ethyl-N1-(2-methoxy-benzyl)-butane-1,4-diamine (3) (0.43 g; 1.82 mmol) in 10 ml of cold EtOH is added dropwise to a suspension of 2,5 dimethoxybenzoquinone (0.16 g; 0.91 mmol) in boiling EtOH (10 ml). It is left under agitation at 55-60° C. for 6 hours, and then the solution is cooled and filtered using a folded filter. The solution evaporated under vacuum provides the required product in the form of solid EtOH crystallised several times. Yield 72%; red solid; m.p. 97° C. ¹H NMR (free base, CDCl₃) δ: 1.08 (t, 6H); 1.51-1.77 (m, 8H); 2.42-2.63 (m, 8H); 3.11 (q, 4H); 3.60 (s, 4H); 3.81 (s, 6H); 5.28 (s, 2H); 7.72 (broad t, 2H exchangeable with D₂O); 6.82-7.01 (m, 4H); 7.19-7.23 (m, 2); 7.38-7.42 (m, 2). MS (ESI⁺) m/z=577 (M+H)⁺.

### EXAMPLE 26

### Synthesis of 2,5-bis-{2-[ethyl-(2-methoxy-benzyl)-amine]-pentyl-amine}-[1,4]-benzoquinone (26)

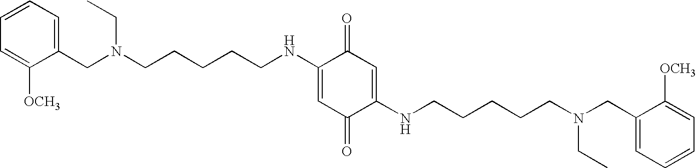

This was synthesised from N1-Ethyl-N1-(2-methoxy-benzyl)-pentane-1,5-diamine (6), following the procedure described in Example 25. Yield 65%; red solid; m.p. 88° C. ¹H NMR (free base, CDCl₃) δ: 1-18 (t, 6H); 1.28-1.72 (m, 12H); 2.43-2.61 (m, 8H); 3.15 (q, 4H); 3.62 (s, 4H); 3.84 (s, 6H); 5.33 (s, 2H); 6.61 (broad t, 2H exchangeable with D₂O); 6.86-7.01 (m, 4H); 7.22-7.29 (m, 2H); 7.42-7.46 (m, 2H). MS (ESI⁺) m/z=605 (M+H)⁺.

### EXAMPLE 27

### Synthesis of 2,5-bis-{6-[ethyl-(2-methyl-benzyl)-amine]-hexylamine}-[1,4]-benzoquinone (27)

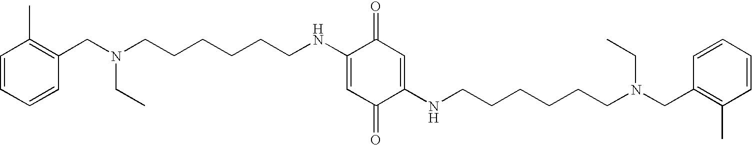

This is obtained by treating N1-Ethyl-N1-(2-methyl-benzyl)-1,6-hexanediamine (22)(0.60 g; 2.42 mmol) with 2,5 dimethoxybenzoquinone as described in Example 25; red waxy solid. ¹H NMR (free base, CDCl₃) δ: 1.03 (t, 6H); 1.22-1.73 (m, 1 GH); 2.28-2.58 (m, 14H); 3.12 (q, 4H); 3.55 (s, 4H); 5.32 (s, 2H); 6.61 (broad t, 2H exchangeable with D₂O); 7.12-7; 21 (m, 6H); 7.25-7.40 (m, 2H). MS (ESI⁺) m/z=601 (M+H)⁺.

### EXAMPLE 28

### Synthesis of 2,5-bis-[6-(ethyl-thiophen-2-ylmethyl-amine)-hexylamine]-[1,4]benzoquinone (28)

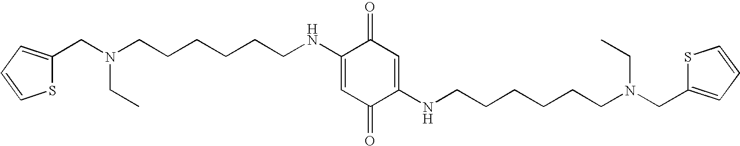

This was synthesised from N¹-ethyl-N¹-thiophen-2-ylmethyl-1,6-hexanediamine (9) (0.44 g; 1.8 mmol) and 2,5 dimethoxybenzoquinone, following the procedure described in Example 25. Yield 85%; red solid; m.p. 55° C. ¹H NMR (free base; CDCl₃) 6; 1.08 (t, 6H); 1.35-1.75 (m, 16H); 2.41-2.62 (m, 8H); 3.18 (q, 4H); 3.81 (s, 4H); 5.38 (s, 2H); 6.62 (broad s, 2H exchangeable with D₂O); 6.88-7.00 (m, 4H); 7.21-7.25 (m, 2H). MS (ESI⁺) m/z 585 (M+H)⁺.

### EXAMPLE 29

### Synthesis of 2,5-bis-[6-(ethyl-pyridin-2-ylmethyl-amine)-hexylamine]-[1,4]benzoquinone (29)

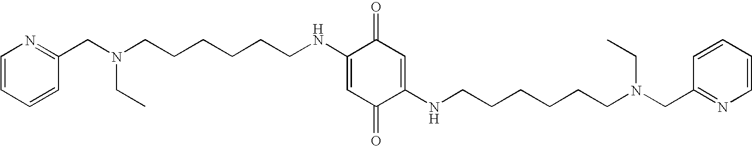

This was synthesised from N¹-ethyl-N¹-pyridin-2-ylmethyl-hexane-1,6-diamine (12) (0.35 g; 1.42 mmol) and 2,5 dimethoxybenzoquinone, following the procedure described in Example 25. Yield 65%; red waxy solid. ¹H NMR (free base; CDCl₃) δ: 1.02 (t, 6H); 1.18-1.68 (m, 16H); 2.38-2.60 (m, 8H); 3.12 (q, 4H); 3.64 (s, 4H); 5.22 (5, 2H); 6.60 (broad s, 2H exchangeable with D₂O); 7.08-7.17 (m, 2H); 7.40-7.48 (m, 2H); 7.57-7.68 (m, 2H); 8.42-8.48 (m, 2H). MS (ESI⁺) m/z=575 (M+H)⁺.

### EXAMPLE 30

### Synthesis of 2,5-bis-[6-(ethyl-pyridin-3-ylmethyl-amine)-hexylamine]-[1,4]benzoquinone (30)

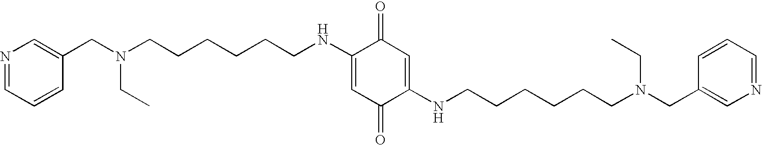

This was synthesised from N¹-ethyl-N¹-pyridin-3-ylmethyl-hexane-1,6-diamine (15) (0.35 g; 1.42 mmol) and 2,5 dimethoxybenzoquinone, following the procedure described in Example 25. Yield 60%; red waxy solid. ¹H NMR (free base; CDCl₃) δ: 1.02 (t, 6H); 1.22-1.66 (m, 16H); 2.33-2.56 (m, 8H); 3.12 (q, 4H); 3.53 (s, 4H); 5.25 (s, 2H); 6.62 (broad s, 2H exchangeable with D₂O); 7.19-7.24 (m, 2H); 7.62-7.68 (m, 2H); 7.57-7.68 (m, 2H); 8.41-8.55 (m, 2H). MS (ESI⁺) m/z=575 (M+H)⁺.

### EXAMPLE 31

### Synthesis of 2,5-bis-[6-(ethyl-pyridin-4-ylmethyl-amine)-hexylamine]-[1,4]benzoquinone (31)

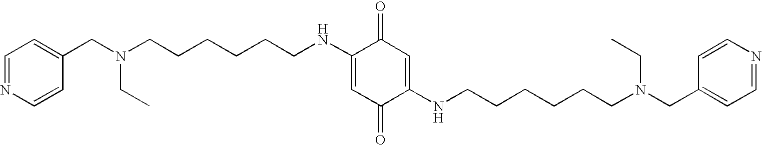

This was synthesised from N¹-ethyl-N¹-pyridin-4-ylmethyl-1,6-hexanediamine (18)(0.40 g; 1.62 mmol) and 2,5 dimethoxybenzoquinone, following the procedure described in Example 25. Yield 70%; red waxy solid. ¹H NMR (free base; CDCl₃) δ: 0.98 (t, 6H); 1.22-1.75 (m, 16H); 2.35-2.58 (m, 8H); 3.12 (q, 4H); 3.52 (s, 4H); 5.25 (s, 2H); 6.63 (broad s, 2H exchangeable with D₂O); 7.21-7.28 (m, 4H); 8.42-8.48 (m, 2H). MS (ESI⁺) m/z=575 (M+H)⁺.

### EXAMPLE 32

### Synthesis of 2,5-bis-{6-[(3,5-dibromine-2-methoxybenzyl)-ethyl-amine]-hexylamine}-[1,4]benzoquinone (32)

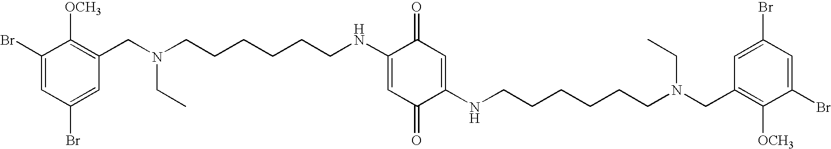

This was synthesised from N¹-ethyl-N¹-(3,5-dibromine-2-methoxy-benzyl)-1,6-hexanediamine (39) (0.10 g; 0.26 mmol) and 2,5 dimethoxybenzoquinone, following the procedure described in Example 25. Yield 80%; red waxy solid. ¹H NMR (free base; CDCl₃) δ: 0.97 (t, 6H); 1.22-1.90 (m, 16H); 2.30-2.58 (m, 8H); 3.08 (q, 4H); 3.58 (s, 4H); 3.85 (s. 6H), 5.35 (s, 2H); 6.62 (broad s, 2H exchangeable with D₂O); 7.55-7.64 (m, 4H). MS (ESI⁺) m/z=949 (M+H)⁺.

### EXAMPLE 33

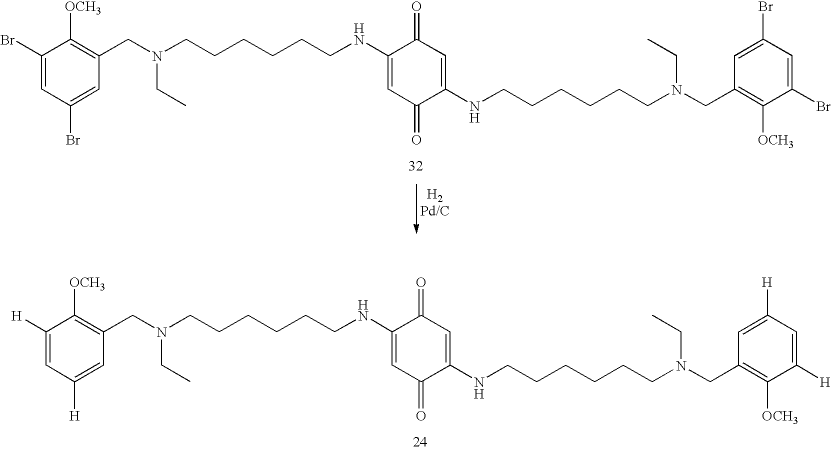

Pd/C at 30% (48 mg) is added to a solution of 2,5-bis-{6-[(3,5-dibromine-2-methoxybenzyl)-ethyl-amine]-hexylamine}[1,4]benzoquinone (32) (60 mg, 0.06 mmol) and sodium acetate (144 mg) in freezing acetic acid (8 ml) and subjected to catalytic hydrogenation until the required theoretical quantity of H₂ has been consumed. The catalyst is filtered through Celite and the colourless solution obtained turns to red within a few minutes in contact with the air. It is basified with sodium carbonate and extracted using CHCl₃ (2 X). The combined organic extracts are anhydrified and concentrated to produce a raw product that is purified through drop chromatography. Elution with CH₂Cl₂ 9.25/MeOH 0.75/NH₄OH 0.075 produces 24 with a yield of 55%.

It can be easily seen that using the present method, it is possible to obtain marked compounds (possibly even tritiated) belonging to the general formula (I) as defined above.

### EXAMPLE 34

### Synthesis of N¹-ethyl-N¹-(2-methoxy-benzyl)-hexane-1,6-diamine (34)

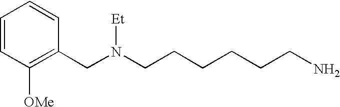

The compound 34 was obtained as a transparent oil from {6-[ethyl-(2-methoxy-benzyl)-amine]-hexyl}-carbamic acid benzyl ester (19) (2.31 g; 6.01 mmol) following the same procedure described in Example 3. Yield 98%; ¹H NMR (free base; CDCl₃) δ: 1.04 (t, 3H), 1.12-1.48 (m, 8H+2H exchangeable with D₂O), 2.41-2.53 (m, 4H), 2.65 (t, 2H), 3.57 (s, 2H), 3.81 (s, 3H), 6.82-6.94 (m, 2H), 7.16-7.42 (m, 2H).

### EXAMPLES 35-36

The Examples 35 and 36 follow the synthetic layout shown below.

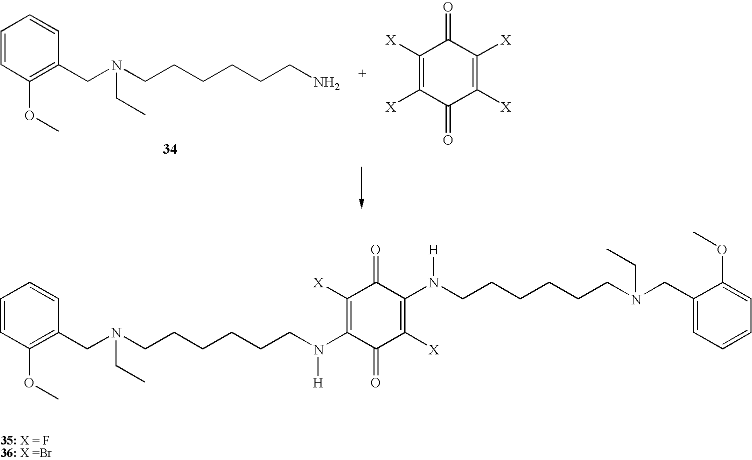

### EXAMPLE 35

### 2,5-bis-{6-[ethyl-(2-methoxy-benzyl)-amine]-hexylamine}-3,6-difluorine-[1,4]benzoquinone (35)

A suspension of tetrafluorine-[1,4]benzoquinone (100 mg; 0.56 mmol) in 2 ml of ether is added dropwise to a solution of N¹-ethyl-N¹-(2-methoxybenzyl)-1,6-hexanediamine (1.23 g; 4.66 mmol) in 3 ml of ethyl ether. The solution immediately becomes a dark red colour. It is left under agitation at room temperature for one hour and all the solvent is evaporated under vacuum. The residue obtained is purified using flash chromatography. Elution with CH₂Cl₂/MeOH/NH₃ aqueous 28% (9:1:0.05) provides the compound 35 in the form of a greenish oil with a yield of 90%.

¹H NMR (free base, CDCl₃) δ: 1.08 (t, 6H); 1.23-1.63 (m complex, 16H); 2.46-2.64 (m, 8H); 3.51 (q, 4H); 3.64 (s, 4H); 6.17 (br s exchangeable with D₂O, 2H); 6.87 (d, 2H); 6.95 (t, 2H); 7.24 (t, 2H); 7.43 (d, 2H).

### EXAMPLE 36

### 2,5-bis-{6-[ethyl-(2-methoxy-benzyl)-amine]-hexylamine}-3,6-dibromine-[1,4]benzoquinone (36)

This was obtained by treating N¹-ethyl-N¹-(2-methoxybenzyl)-1,6-hexanediamine (0.21 g; 0.8 mmol) with tetrabromine-[1,4]benzoquinone (0.17 g; 0.4 mmol) as described in Example 35. Yield 25%; purple oil.

¹H NMR (free base, CDCl₃) δ: 1.07 (t, 6H); 1.28-1.78 (m complex, 1 GB); 2.43-2.60 (m, 8H); 3.60 (s, 4H); 3.84-3.94 (m, 10H, 6.17 (br s, 2H); 6.87 (d, 2H); 6.95 (t, 2H); 7.19-7.28 (m, 2H+2H exchangeable with D₂O); 7.42 (d, 2H). MS (ESI⁴) m/z 791 (M+H)⁺

### EXAMPLE 37

This Example describes the determination of the inhibiting ability in relation to AChE and BuChE and the selectivity of compounds 35 and 36 and the comparison of these properties with certain pharmaceuticals present on the market. The inhibiting power (IC₅₀) was determined using the Ellman spectrophotometric method (Ellman, G. L.; Courtney, K. D.; Andres, V.; Featherstone, R. M. A New and Rapid Colorimetric Determination of Acetylcholinesterase Activity. Biochem. Pharmacol. 1961, Vol. 7, pages. 88-95).

The level of the IC₅₀ was determined using constant concentrations of substratum and enzymes and varying the concentration of an inhibitor with successive increases. In this manner the activity of the enzyme is determined as a revelation of the forming of anionic coloured molecular species (2-nitro 4-thiobenzoate) (λₘₐₓ=412 nm), which is obtained following the reaction between thiocholine—product of the enzymatic hydrolysis of acethyltiocholine (substratum of AChE) or Butyrylcholine (substratum of BuChE)—and a 5,5′ dithiobisnitrobenzoic acid (Ellman reactive). The absorbency variation at 412 nm (in other words the absorbency variation of the 2-nitro 4-thiobenzoate per minute (AA/min) (enzymatic speed) depends on the substratum concentration and on the enzymatic activity of the AChE and BuChE, according to Michaelis Menten kinetics.

In order to calculate the IC₅₀, constant concentrations of saturating substratum were used, that is, those capable of producing maximum enzymatic speed (Vₘₐₓ) and fixed enzyme aliquot. Tests were then made on the increasing concentrations of the compounds under research capable of inducing inhibitions between 20% and 80% of the Vₘₐₓ. Later, inhibition lines were obtained, drawing up a graph of the inhibition percentage of the Vₘₐₓ according to the function of the decimal logarithm of the nanomolar concentration of the inhibitor. Linear regression parameters and the IC₅₀ were estimated for each line (the concentration able to deactivate the maximum enzymatic activity by 50%) which was obtained through interpolation on the relevant line.

TABLE I

| | IC_{50 (AChE)} | IC_{50 (BuChE)} | Selectivity |
|---|---|---|---|
| Inhibitor | (nM) | (nM) | IC_{50 (BuChE)}/IC_{50 (AchE)} |
| 35 | 5.17 ± 0.17 | 1520 ± 90 | 294 |
| 36 | 2.66 ± 0.19 | 800 ± 62 | 300 |
| Tacrine | 250 ± 10 | 50 ± 2 | 0.2 |
| Donepezil | 23.1 ± 4.8 | ~7000 | ~300 |

Table I shows the IC₅₀ obtained using the method described above and relative to the compounds 35, 36, to tacrine and to donepezil. The IC₅₀ relative to AChE are indicated as IC_{50 (AChE)}, the IC₅₀ relative to BuChE indicated as IC_{50 (BuChE)}. The selectivity is defined as the ratio between IC_{50 (AChE)} and IC_{50 (BuChE)} and indicates the ability of the inhibitor to bond preferably with AChE rather than with BuChE.

The compounds 35 and 36 have shown an IC_{50 (AChE)} that is relatively very good (Table I), two orders lower than the IC₅₀(AChE) of the tacrine (Cognex®) and considerably lower than that of donepezil (Aricept®).

The selectivity results as relatively very good, better than that of tacrine and comparable to that of donepezil.

### EXAMPLE 38

### Synthesis of [6-(3,5-dibromine-2-methoxybenzyl)-amine]-hexyl-carbamic acid hydrochloridic benzyl ester (37)

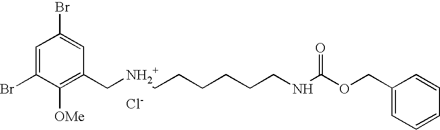

3,5-dibromine-2-methoxybenzaldehyde (1.90 g; 6.4 mmol) is added to a solution of 23 (1.46 g; 5.8 mmol) in 60 ml of ethanol containing molecular sieving. After 30 minutes NaBH₄ (0.23 g; 6.2 mmol) is added and the reaction mixture is left under agitation overnight. Acidification with HCl 6 N provides the precipitation of a solid that is collected by filtration and washed repeatedly with ether. Yield 65%.

¹H NMR (hydrochloride salt; CDCl₃) δ: 1.22-1.98 (m, 8H); 2.75-2.95 (m, 2H); 3.08 (t, 2H); 3.94 (s, 3H); 4.12 (s, 2H); 5.04 (s, 2H); 7.38 (s, 5H); 7.73-7.75 (m, 1H); 7.99-8.01 (m, 1H); 9.57 (broad s, 2H exchangeable with D₂O)

### EXAMPLE 39

### Synthesis of {6-[ethyl-(3.5-dibromine-2-methoxybenzyl)-amine]-hexyl}-carbamic acid benzyl ester (38)

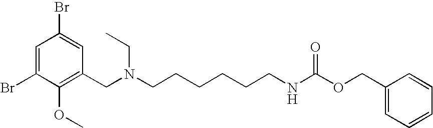

This compound was prepared following the procedure in Example 19 of [6-(3,5-dibromine-2-methoxybenzyl)-amine]-hexyl-carbamic acid hydrochloric benzyl ester (37) (2.09 g; 3.7 mmol) and acetaldehyde (0.45 ml; 7.9 mmol). The raw product obtained is purified using flash chromatography with mobile phase using petroleum ether/CH₂Cl₂/acetone/NH₃ aqueous 28% (8:1:1:0.05); yellow oil. in NMR (free baser CDCl₃) δ: 1.04 (t, 3H); 1.20-1.59 (m, 8H); 2.35-2.58 (m, 4H); 3.19 (q, 2H); 3.58 (s, 2H); 3.81 (s, 3H); 4; 82 (broad t, 1H exchangeable with D₂O); 5.16 (s, 2H); 7.15-7.21 (m, 3H); 7.28-7.41 (m, 6H).

### EXAMPLE 40

### Synthesis of N¹-ethyl-N¹-(3,5-dibromine-2-methoxy-benzyl)-1,6-hexanediamine (39)

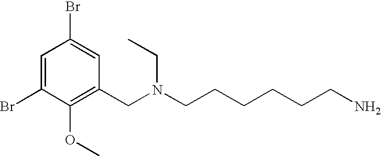

It was obtained by treating the compound 38 (017 g; 0.30 mmol) with HBr as described in Example 3. Yield 87%; yellow oil.

¹H NMR (free base, CDCl₃) δ 1.05 (t, 3H); 1.20-1.58 (m, 8H) 2.05 (broad s, 3H exchangeable with D₂O); 2.41 (t, 2H); 2.56 (q, 2H); 2.63-2.78 (m, 2H); 3.60 (s, 2H); 3.81 (s, 3H); 7.57-7.63 (m, 2H).

1. Method for the synthesis of a 2,5-bis-diamine-[1,4]benzoquinonic derivative having a general formula (I): 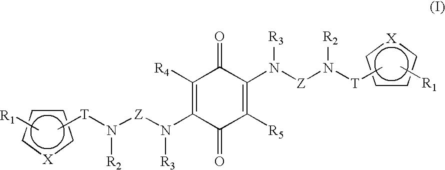
wherein R₁ represents a substituent chosen in the group consisting of:
a hydrogen,
a saturated or unsaturated, linear or branched alkyl group with one to five carbon atoms, and,
a substituent having an inductive electron withdrawing effect with comparison to hydrogen;
R₂ and R₃ represent, each independently from one another, hydrogen, or a saturated or unsaturated, linear or branched alkyl group with one to five carbon atoms; R₄ and R₅ represent each independently from one another, a substituent chosen in the group consisting of:
hydrogen,
a saturated or unsaturated, linear or branched alkyl group with one to five carbon atoms,
a halogen,
X represents a radical chosen in the group consisting of: —HC═CH—, —HC═N—, —S—, —O—, and —NH—;
T represents a saturated or unsaturated, linear or branched alkyl group with one to four carbon atoms;
Z represents a saturated or unsaturated, linear or branched alkyl with two to thirteen carbon atoms;
the method being characterised in that it comprises a nucleophilic substitution phase, wherein on a p-benzoquinone having a general formula (IX): 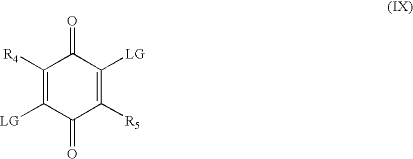
wherein LG represents a leaving group chosen in the group consisting of: an alkoxy group C₁-C₅ and a halogen, a substitution is performed with a first intermediate compound having a general formula (VIII): 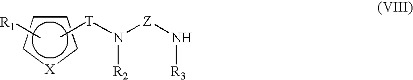
in order to obtain the 2,5-bis-diamine-[1,4]benzoquinonic derivative having a general formula (I).

2. Method according to claim 1, wherein R₁ represents a substituent chosen in the group consisting of:
a hydrogen,
a substituent having an inductive electron withdrawing effect.

3. Method according to claim 1, wherein R₁ represents a substituent chosen in the group consisting of:
a hydrogen,
a halogen,
NO₂, and
an alkoxy C₁-C₃;
R₂ and R₃ represent, each independently from one another, a hydrogen or a saturated or unsaturated linear alkyl group having from one to four carbon atoms; R₄ and R₅ represent, each independently from one another, a substituent chosen in the group consisting of:
hydrogen,
a saturated or unsaturated, linear or branched alkyl group with one to five carbon atoms,
a halogen,
T represents a saturated or unsaturated, linear alkyl with one to three carbon atoms;
Z represents a saturated or unsaturated, linear alkyl with two to twelve carbon atoms.

4. Method according to claim 1, wherein R₁ represents a substituent chosen in the group consisting of:
a hydrogen,
a halogen,
a saturated or unsaturated linear or branched alkyl group having from one to four carbon atoms, and
an alkoxy C₁-C₃;
R₂ and R₃ represent, each independently from one another, a hydrogen or a saturated linear alkyl group having one to two carbon atoms; R₄ and R₅ represent, each independently from one another, a substituent chosen in the group consisting of:
hydrogen,
a saturated linear or branched alkyl group having from one to four carbon atoms,
a halogen,
X represents the radical —HC═CH— or the radical —O—;
T represents the radical —CH2-; and
Z represents a saturated linear alkyl having from two to seven carbon atoms.

5. Method according to claim 1, wherein R₃ represents a hydrogen; R₄ and R₅ represent, each independently from one another, a substituent chosen in the group consisting of
hydrogen,
a saturated linear alkyl group having from one to two carbon atoms,
a branched alkyl (group having from three to four carbon atoms, a halogen,
Z represents a saturated linear alkyl having from two to seven carbon atoms.

6. Method according, to claim 1, wherein R₁ is in position 2 compared to T.

7. Method according to claim 1, wherein X represents a radical chosen in the group consisting of: —HC═CH—, —HC═N— and —S—.

8. Method according to claim 7, wherein X represents the radical —HC═CH—.

9. Method according to claim 1, wherein R₄ and R₅ each represent a respective hydrogen.

10. Method according to claim 1, wherein R₄ and R₅ each represent a respective halogen, preferably a fluorine or a bromine.

11. Method according to claim 1, wherein R₂ represents a saturated linear alkyl having from one to two carbon atoms; R₃ represents a hydrogen; R₁ represents an alkoxy group C₁-C₂.

12. Method according to claim 1, wherein R₁ represents a methoxy group, R₂ represents an ethyl, R₃, R₄ and R₅ each represent a relative hydrogen, Z represents a hexyl, T represents a methyl, X represents the radical —HC═CH—.

13. Method according to claim 1, wherein R₁ represents a methoxy group, R₂ represents an ethyl, R₃, R₄ and R₅ each represent a relative hydrogen, Z represents a propyl, T represents a methyl, X represents the radical —HC═CH—.

14. Method according to claim 1, wherein R₁ represents a methoxy group, R₂ represents an ethyl, R₃, R₄ and R₅ each represent a relative hydrogen, Z represents a butyl, T represents a methyl, X represents the radical —HC═CH—.

15. Method according to claim 1, wherein R₁ represents a methoxy group, R₂ represents an ethyl, R₃, R₄ and R₅ each represent a relative hydrogen, Z represents a pentyl, T represents a methyl, X represents the radical —HC═CH—.

16. Method according to claim 1, wherein R₁ represents a methyl, R₂ represents an ethyl, R₃, R₄ and R₅ each represent a relative hydrogen, Z represents a hexyl, T represents a methyl, X represents the radical —HC═CH—.

17. Method according to claim 1, wherein R₁ represents a methoxy group, R₂ represents an ethyl, R₃, R₄ and R₅ each represent a relative hydrogen, Z represents a hexyl, T represents a methyl, X represents the radical —S—.

18. Method according to claim 1, wherein R₁ represents a methoxy group, R₂ represents an ethyl, R₃, R₄ and R₅ each represent a relative hydrogen, Z represents a hexyl, T represents a methyl, X represents the radical —HC═N—.

19. Method according to claim 1, wherein R₁ represents a methoxy group, R₂ represents an ethyl, R₃ represents a hydrogen, R₄ and R₅ each represent a relative fluorine, Z represents a hexyl, T represents a methyl, X represents the radical —HC═CH—.

20. Method according to claim 1, wherein R₁ represents a methoxy group, R₂ represents an ethyl, R₃ represents a hydrogen, R₄ and R₅ each represent a relative bromine, Z represents a hexyl, T represents a methyl, X represents the radical —HC═CH—.

21. Method according to claim 1, wherein LG represents an alkoxy group.

22. Method according to claim 1, wherein LG represents an alkoxy group C₁-C₅.

23. Method according to claim 1, wherein LG represents an alkoxy group C₁-C₃.

24. Method according to claim 1, wherein LG represents a methoxy group.

25. Method according to claim 1, wherein LG represents a halogen.

26. Method according to claim 1, wherein LG represents a halogen chosen in the group consisting of: fluorine, bromine.

27. Method according to claim 1, wherein the nucleophilic substitution phase occurs in the presence of an alcoholic solvent.

28. Method according to claim 27, wherein the alcoholic solvent is ethanol.

29. Method according to claim 1, wherein the nucleophilic substitution phase occurs at a temperature between 50° C. and 65° C.

30. Method according to claim 1, and comprising a hydrolysis phase: wherein a second intermediate compound having a general formula (VII): 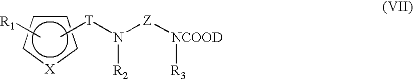
wherein D represents a benzyl (Bn) or another protective group that is basically stable in a base environment, is hydrolysed in order to obtain said first intermediate compound having the general formula (VII).

31. Method according to claim 30, and comprising an addition phase, wherein a third intermediate compound having a general formula (V): 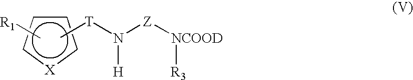
is made to react in presence of a reducer with a compound having a general formula (VI):
R₂═O (VI),
in order to obtain said second intermediate compound having a general formula (VII).

32. Method according to claim 31, wherein the reducer is NaBH3CN.

33. Method according to claim 31, wherein the addition phase occurs in an alcoholic solvent.

34. Method according to claim 31, wherein the compound having the general formula (VI) is an aldehyde.

35. Method according to claim 30, and comprising an addition phase, wherein a third intermediate compound having the general formula (V): 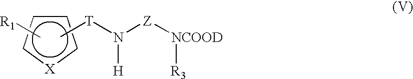
is made to react in the presence of a reducer with a compound having a general formula (XIII):
R₂COOH (XIII),
in order to obtain the said second intermediate compound having the general formula (VII).

36. Method according to claim 35, wherein the reducer is NaBH4.

37. Method according to claim 35, wherein the addition phase occurs in Tetrahydrofuran (THF) as the solvent.

38. Method according to claim 31, wherein the third intermediate compound having the general formula (VII) is purified through extraction with a basically apolar solvent.

39. Method according to claim 31, and comprising a second addition phase, wherein a protected (amine-alkyl)- 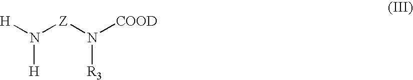
carbamic acid having the general formula (III):
is added to a compound having the general formula (IV): 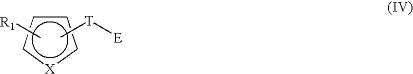
wherein E represents a residue chosen in the group consisting of: ═O —Cl, —Br, and —I; in order to obtain the said third intermediate compound having the general formula (V); with the condition that where E represents ═O, the method comprises a reduction phase to obtain said third intermediate compound having the general formula (V).

40. Method according to claim 39, wherein E represents ═O.

41. Method according to claim 39, and comprising a protection phase, wherein a diamine having the general formula (II): 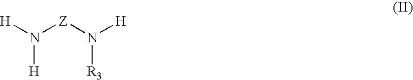
is made to react with benzylchloroformiate in order to obtain protected (amine-alkyl)-carbamic acid having the general formula (III) wherein D represents a benzyl (Bn).

42. Method according to claim 41, wherein the molar ratio between the (amine-alkyl)-carbamic acid having the general formula (II) and the benzylchloroformiate is approximately three to one.

43. Method for the synthesis of a second intermediate compound having the general formula (VII), comprising an addition phase as defined in claim 31.

44. 2,5-bis-diamine-[1,4]benzoquinonic derivative having the general formula (X): 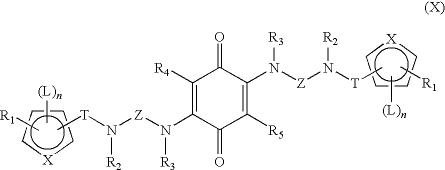
wherein R₁, R₂, R₃, R₄, R₅, X, T and Z are defined as in claim 1, L represents a bromine and n is an integer greater than or equal to 1 and less than or equal to 3.

45. Derivative according to claim 44, wherein L represents a bromine and n is equal to 2.

46. Method for the synthesis of a 2,5-bis-diamine-[1,4]benzoquinonic derivative having the general formula (I) as defined in the method comprising the reduction phase of a 2,5-bis-diamine-[1,4]benzoquinonic derivative having the general formula (X) according to claim 44.

47. Method according to claim 46, wherein during the reduction phase the 2,5-bis-diamine-[1,4]benzoquinonic derivative having the general formula (I) is subjected to catalytic hydrogenation.

48. Method for the synthesis of a compound having the general formula (X) as defined claim 44; comprising a nucleophilic substitution phase, wherein on a p-benzoquinone having the general formula (IX): 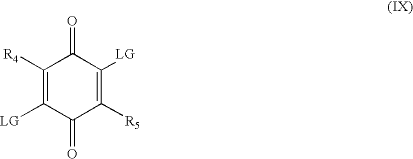
wherein LG represents a leaving group having an inductive electron withdrawing effect, a substitution is performed with a first intermediate compound having the general formula (XI): 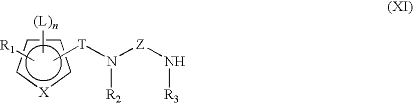
in order to obtain the compound having the general formula (X) as defined in claim 44.

49. Method according to claim 48, wherein LG is defined as in claim 21.

50. Method according to claim 48, wherein the nucleophilic substitution phase is produced as defined in claim 27.

51. 2,5-bis-diamine-[1,4]benzoquinonic derivative having the general formula (XII): 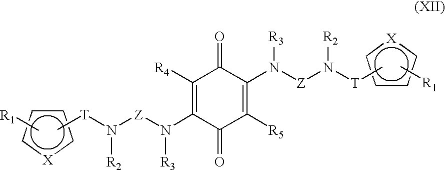
wherein R₄ and R₅ each represent, a respective halogen; R₁, R₂, R₃, X, T and Z being defined as in claim 1.

52. Derivative according to claim 51, wherein R4 and R5 each represent a respective halogen chosen in the group consisting of: Fluorine, Chlorine and Bromine.

53. Derivative according to claim 51, wherein R₄ and R₅ represent a halogen chosen in the group consisting of: fluorine and bromine.

54. (canceled)

55. (canceled)

56. A medication comprising a derivative according to claim 51.

57. A method of treating Alzheimer's disease comprising administering a therapeutically effective amount of a derivative of claim 51 to an individual in need thereof.
